(19) United States
(12) Patent Application Publication
(10) Pub. No.: US 2010/0173990 A1
(43) Pub. Date: Jul. 8, 2010

(54) Transglutaminase Inhibitor Comprising Chlorogenic Acid And A Method For Producing Thereof

(76) Inventors: Soo Youl Kim, Seoul (KR); Kyung Chae Jeong, Gyeonggi-do (KR)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/663,348

(22) PCT Filed: Jun. 11, 2008
(86) PCT No.: PCT/KR2008/003264

§ 371 Date: Dec. 29, 2009

(30) Foreign Application Priority Data
Jun. 11, 2007 (KR) 10-2007-0056811

(51) Int. Cl. A61K 31/216; C07C 69/732; A61P 35/00; A61P 25/28; A61P 25/16

(52) U.S. Cl. 514/533; 560/75

(57) ABSTRACT

The present invention relates to a transglutaminase inhibitor comprising chlorogenic acid, a pharmaceutically acceptable salt thereof, or a derivative thereof. More particularly, the present invention relates to a transglutaminase inhibitor comprising chlorogenic acid, a pharmaceutically acceptable salt thereof, or a derivative thereof which effectively inhibits the activity of transglutaminase, the overexpression of which is responsible for the etiology of various diseases, and to novel uses thereof. The present invention provides a transglutaminase inhibitor and a method of inhibiting transglutaminase, both of which are based on using chlorogenic acid, a pharmaceutically acceptable salt thereof, or a derivative thereof as an active ingredient. Featuring the use of chlorogenic acid, a pharmaceutically acceptable salt thereof, or a derivative thereof, the novel method of inhibiting transglutaminase according to the present invention is safely applied to patients who suffer from the diseases caused by the overexpression of transglutaminase, thereby obtaining an inhibitory effect against transglutaminase without causing side-effects.

### TECHNICAL FIELD

The present invention relates to a transglutaminase inhibitor comprising chlorogenic acid, a pharmaceutically acceptable salt thereof, or a derivative thereof. More particularly, the present invention relates to a transglutaminase inhibitor comprising chlorogenic acid, a pharmaceutically acceptable salt thereof, or a derivative thereof which effectively inhibits the activity of transglutaminase, the overexpression of which is responsible for the etiology of various diseases, and to novel uses thereof.

### BACKGROUND ART

Transglutaminases are protective enzymes which are responsible for blood clotting in response to tissue injury under normal conditions. However, these enzymes are also reported to play an important role in the pathological mechanism of various diseases in the absence of regulatory-control in the level of expression thereof (review article. Soo-Youl Kim: New Target Against Inflammatory Diseases: Transglutaminase 2. Archivum Immunologiae & Therapiae Experimentalis 52, 332-337, 2004).

The expression of transglutaminases increases particularly upon the occurrence of various inflammatory diseases, including rheumatoid arthritis, diabetes, inflammatory myositis, atherosclerosis, stroke, liver cirrhosis, breast cancer, Alzheimer's disease, Parkinson's disease, Huntington's disease, encephalitis, and celiac disease. Also, transglutaminases are observed to increase in expression level, along with NF-κB, when cancer enters metastasis or changes into chemo-resistance or radio-resistance (Soo-Youl Kim. Transglutaminase 2 in inflammation. Front Biosci. 11, 3026-3035, 2006).

The relationship between transglutaminases and chemo-resistance in cancer has remained unclear so far. However, when the expression of transglutaminases was suppressed in chemoresistant breast cancer cells, the cancer cells were getting highly susceptible to chemicals, and finally died (Antonyak et al., Augmentation of tissue transglutaminase expression and activation by epidermal growth factor inhibit doxorubicin-induced apoptosis in human breast cancer cells. J Biol Chem. 2004 Oct. 1; 279(40):41461-7.; Dae-Seok Kim et al. Reversal of Drug Resistance in Breast Cancer Cells by Transglutaminase 2 Inhibition and Nuclear Factor-KB Inactivation. Cancer Res. 2006. in press).

Also, there is a strong reason for suppressing the activity of transglutaminases as the etiological mechanism for which the activation of transglutaminases is responsible is elucidated at the molecular level (Key Chung Park, Kyung Cheon Chung Yoon-Seong Kim, Jongmin Lee, Tong H. Joh, and Soo-Youl Kim. Transglutaminase 2 induces nitric oxide synthesis in BV-2 microglia. Biochem. Biophys. Res. Commun. 323, 1055-1062, 2004; Jongmin Lee, Yoon-Seong Kim, Dong-Hee Choi, Moon S. Bang Tay R. Han, Tong H. Joh, and Soo-Youl Kim. Transglutaminase 2 induces NF-κB activation via a novel pathway in BV-2 microglia. J. Biol. Chem. 279, 53725-53735, 2004; Dae-Seok Kim et al. Reversal of Drug Resistance in Breast Cancer Cells by Transglutaminase 2 Inhibition and Nuclear Factor-KB Inactivation. Cancer Res. 2006. in press).

Inflammation is largely attributable to NF-κB activation. NF-κB is known to be activated by kinases in signal transduction pathways. However, NF-κB was also found to be activated independently of kinases, thereby negating the function of kinase inhibitors (Tergaonkar et al., IkappaB kinase-independent IkappaBalpha degradation pathway: functional NF-kappaB activity and implications for cancer therapy. Mol Cell Biol. 2003 November; 23(22):8070-83.).

In a previous study conducted by the present inventors, it was reported that transglutaminase activates NF-κB independently of the activation of kinases (IKK, NAK), by inducing crosslinking I-κBa (Jongmin Lee, et al. Transglutaminase 2 induces NF-kB activation via a novel pathway in BV-2 microglia. J. Biol. Chem. 279, 53725-53735, 2004). Transglutaminases are calcium-dependent enzymes, which can activate NF-κB only at an elevated intracellular level of calcium.

Upon inflammation, the activation of the transcriptional factor NF-κB leads to an increase in the expression not only of inflammatory factors including transglutaminases, but also of its inhibitor I-κBα. Continuous NF-κB activation is inhibited by I-κBα under normal conditions, but continues in chronic inflammatory diseases. Interestingly, TNF-α or LPS (lipopolysaccharide)-induced NF-κB activation gives rise to transglutaminase expression. Thus, aberrantly activated transglutaminases in inflammatory cells are expected to activate NF-κB directly or to further maintain activated NF-κB, thereby playing a key role in inflammation maintenance (FIG. 1). In addition, this vicious cycle may be a main cause of cancer metastasis and chemoresistance (Jongmin Lee, et al. Transglutaminase 2 induces NF-κB activation via a novel pathway in BV-2 microglia. J. Biol. Chem. 279, 53725-53735, 2004).

Therefore, a transglutaminase inhibitor may play a crucial role in breaking the continuous cycle of NF-κB, on which the steroid-substituting effect proposed by the present inventors is based (Sohn, J., Kim, T.-I., Yoon, Y.-H., and Kim, S.-Y.: Transglutaminase Inhibitor: A New Anti-Inflammatory Approach in Allergic Conjunctivitis. J. Clin. Invest. 111, 121-8, 2003).

Amine compounds are known to inhibit transglutaminase activity. Representative of the transglutaminase inhibitors are cystamine (nature Genetics 18, 111-117, 1998; Nature Medicine 8, 143-149, 2002) and putrescine. In addition to the amine compounds, other chemical inhibitors, such as monodansylcadaverine (J. Med. Chem. 15, 674-675, 1972), w-dibenzylaminoalkylamine (J. Med. Chem. 18, 278-284, 1975), 3-halo-4,5-dihydroisoxazole (Mol. Pharmacol. 35, 701-706, 1989), and 2-[(2-oxopropyl)thio]imidazolium derivatives (Blood, 75, 1455-1459, 1990), were developed, but are reported to be so toxic as to non-specifically inhibit other enzymes in vivo.

Therefore, there is a need for safe and effective transglutaminase-specific inhibitors. Recently, Sohn et al. have succeeded in obtaining the same effect from recombinant peptides as steroidal drugs for the inflammation of allergic conjunctivitis to ragweed in a guinea pig model (Sohn, J., Kim, T.-I., Yoon, Y.-H., and Kim, S.-Y.: Transglutaminase Inhibitor: A New Anti-Inflammatory Approach in Allergic Conjunctivitis. J. Clin. Invest. 111, 121-8, 2003). In this regard, anti-flammin protein (FLA2 inhibitor) or elafin protein (very strong transglutaminase substrate, Nara, K., et al. 1994. Elastase inhibitor elafin is a new type of proteinase inhibitor which has a transglutaminase-mediated anchoring sequence termed “cementoin”. J Biochem (Tokyo). 115:441-448)-derived synthetic peptides which mimic the catalytic site of transglutaminase were used. The expression of transglutaminases increases particularly upon the occurrence of various inflammatory diseases, including degenerative arthritis, diabetes, autoimmune myositis, arteriosclerosis, cerebral apoplexy, hepatocirrhosis, malignant breast cancer, meningitis, and inflammatory gastric ulcer.

In addition to the above-mentioned compounds, other chemical inhibitors were developed, but are reported to be so toxic as to non-specifically inhibit other enzymes. Effective as they are in inhibiting transglutaminase, peptide inhibitors developed prior to the present invention (Korean Patent Application No. 10-2006-98921) still have a lot of problems awaiting solutions in terms of production cost and safe practice.

On the other hand, chlorogenic acid is an ester of caffeic acid and quinic acid, a major phenolic compound in coffee, and isolated from the leaves and fruits of dicotyledonous plants. Especially, chlorogenic acid is present in the green or roasted coffee bean, and can be readily extracted using a mixed solution of methanol and water by HPLC-UV [Bicchi et al., J. Agric. Food Chem., 43, pp 1549-1555 (1995)]. Chlorogenic acid is a substance found in coffee and Psilantus, and its isolation method and genealogical classification are disclosed by Clifford et al. in Phytochemistry, Vol. 28, No. 3, pp 829-838(1989). This compound is an important factor in plant metabolism, known as an antioxidant, and also known to slow the release of glucose into the bloodstream after a meal.

In addition, a natural or synthesized chlorogenic acid is used for preventing or treating the decline of male reproductive function against hormone-disrupting chemicals (Korean Patent Publication No. 10-2001-87593), and chlorogenic acid extracted from coffee, nandina leaf, or unripe apple fruit is also used for preventing and treating hypertension (Japanese Patent Publication No. 2002-363075). Further, disclosed is a coffee drink composition having an excellent effect of reducing blood pressure, in which chlorogenic acid is contained, and the content of hydroxyhydroquinone is reduced (Korean Patent Publication No. 10-2006-128907). However, there is no mention of its inhibitory activity against transglutaminase in the prior art.

### DISCLOSURE OF INVENTION

### Technical Problem

The present inventors have conducted intensive and thorough research into transglutaminase inhibitors by screening naturally occurring compounds which have been recognized as being safe for commercialization. They found that chlorogenic acid has a potent inhibitory activity against transglutaminase, thereby completing the present invention.

### Technical Solution

It is an object of the present invention to provide a transglutaminase inhibitor comprising chlorogenic acid, a pharmaceutically acceptable salt thereof, or a derivative thereof.

In addition, it is another object of the present invention to provide a method of inhibiting transglutaminase, featuring the use of chlorogenic acid, a pharmaceutically acceptable salt thereof, or a derivative thereof as an active ingredient.

It is still another object of the present invention to provide a pharmaceutical composition for the treatment of diseases caused by the activation of transglutaminase, comprising chlorogenic acid, a pharmaceutically acceptable salt thereof, or a derivative thereof.

It is still another object of the present invention to provide a method of treating diseases caused by the activation of transglutaminase, featuring the use of chlorogenic acid, a pharmaceutically acceptable salt thereof, or a derivative thereof.

### BRIEF DESCRIPTION OF THE DRAWINGS

FIG. 1 is a graph showing the result of in vitro assay for inhibitory effect of chlorogenic acid on transglutaminase, in which the transglutaminase-catalyzed reaction between [1,4,-¹⁴C] putrescine and succinylated casein is measured, resulting in that chlorogenic acid, competing with putrescine, acts to inhibit the activity of transglutaminase.

### BEST MODE FOR CARRYING OUT THE INVENTION

In accordance with an aspect, the present invention relates to a transglutaminase inhibitor comprising chlorogenic acid, a pharmaceutically acceptable salt thereof, or a derivative thereof, and a method of inhibiting transglutaminase, featuring the use of chlorogenic acid as an active ingredient.

As mentioned above, chlorogenic acid is generally present in the leaves or fruits of dicotyledonous plants, and has the chemical name of 3-[[3-(3,4-dihydroxyphenyl)-1-oxo-2-propenyl]oxy]-1,4,5-trihydroxycyclohexanecarboxylic acid. It can be also named as 3-caffeoylquinic acid. A small amount of isomers, such as isochlorogenic acid and neochlorogenic acid, is contained in naturally present chlorogenic acid, and caffeic acid is produced at the time of hydrolysis. Chlorogenic acid has a molecular formula of C₁₆H₁₈0₉, a molecular weight of 354.30, and a

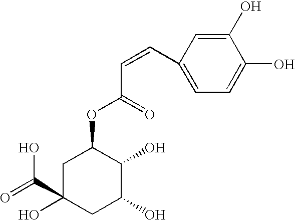

In the present invention, a natural or synthesized chlorogenic acid may be used, and the natural chlorogenic acid may be obtained by purifying or isolating the natural phenol and polyphenol extracted from Rosaceae fruit such as apple, pear and peach, coffee bean, cacao bean, seed of grape, and artichoke. Especially, the preferred origin of chlorogenic acid is green bean or roasted bean of coffee according to a known method (H. Li et al., J. Chromatogr. A 1098(2005) 66-74 and V. Ossipov et al., J. Chromatogr. A 721(1996) 59-68). In addition, the synthesized chlorogenic acid may be obtained by a synthetic method according to a known method (M. Lepelley et al., Plant Science 172(2007) 978-996 and J. Stockigt et al., FEBS LETTERS 42(1974) 131-134). The natural or synthesized chlorogenic acid may be directly prepared or commercially available.

The chlorogenic acid derivative is an ester form isolated from a natural source, known as methyl chlorogenate and ethyl chlorogenate, and U.S. Pat. No. 6,632,459 discloses a chlorogenic acid derivative, in which a hydroxyl group of chlorogenic acid is substituted with caffeic acid. The transglutaminase inhibitor according to the present invention includes the chlorogenic acid derivatives having an inhibitory activity against transglutaminase, which is equivalent to chlorogenic acid.

In addition, the transglutaminase inhibitor according to the present invention includes a pharmaceutically acceptable salt of chlorogenic acid. The salt of chlorogenic acid may be exemplified by sodium chlorogenate, potassium chlorogenate, and magnesium chlorogenate. The preferred salt of chlorogenic acid according to the present invention includes sodium chlorogenate and potassium chlorogenate.

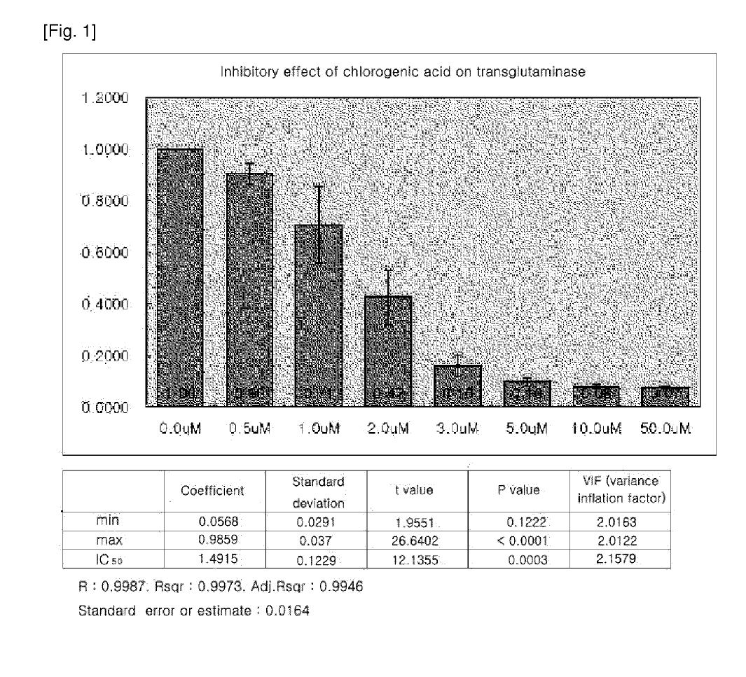

In a specific embodiment of the present invention, the present inventors measured transglutaminase-catalyzed reaction between [1,4,-¹⁴C] putrescine and succinylated casein. It was found that chlorogenic acid, competing with putrescine, acts to inhibit the activity of transglutaminase. In in vitro experiments for transglutaminase-catalyzed reaction between ¹⁴C-labelled putrescine and succinylated casein, it was found that a higher concentration of chlorogenic acid leads to a poorer activity of transglutaminase (FIG. 1). Consequently, it can be seen that chlorogenic acid is a transglutaminase inhibitor, and chlorogenic acid can reduce the increased activity of transglutaminase, even upon the overexpression of transglutaminase.

In accordance with another aspect, the present invention relates to a pharmaceutical composition for the prevention and treatment of diseases caused by the activation of transglutaminase, comprising chlorogenic acid, a pharmaceutically acceptable salt thereof, or a derivative thereof, and to a method of treating the diseases with chlorogenic acid, a pharmaceutically acceptable salt thereof, or a derivative thereof.

The term “prevention” as used herein means all of the actions in which the occurrence of any disease caused by the activation of transglutaminase is restrained or retarded by the administration of the pharmaceutical composition containing chlorogenic acid, a pharmaceutically acceptable salt thereof, or a derivative thereof. The term “treatment” as used herein means all of the actions in which any disease caused by the activation of transglutaminase has taken a turn for the better or been modified favorably by the administration of the pharmaceutical composition.

In the present invention, the diseases caused by the activation of transglutaminase include all diseases that are incurred as transglutaminase activity increases, for example, upon the overexpression of transglutaminase, and are particularly exemplified by neurological diseases and cancers.

Typical of neurological diseases are central nervous system diseases, which are associated with the death or injury of the central nervous system, such as Alzheimer's disease, multi-infarct dementia, a mixed Alzheimer/multi-infarct dementia, Parkinson's disease, hypothyroidism, alcohol-related dementia, and Huntington's diseases. These diseases are characterized by confusion, disorientation and personality disintegration with main syndromes of cognitive dysfunction, language impairment, dysfunctions in judgment, inference, temporal and spatial adaptation and learning finally leading to the death of afflicted patients. Of them, the diseases caused by the activation of transglutaminase, e.g., the overexpression of transglutaminase in nerve tissues, are targets of the pharmaceutical composition according to the present invention. Particularly, the pharmaceutical composition of the present invention is useful in the treatment of Huntington's disease, which is associated with the over-expression of transglutaminase in the brain (Nature Medicine, Vol 8. Number 2, February 2002 pp 143-149), Alzheimer's disease, which is associated with the over-expression of transglutaminase in the cerebellum and cerebral cortex (The Journal of Biological Chemistry, Vol. 274. No. 43. Issue Of October 22, pp 30715-30721), and Parkinson's disease, which is associated with transglutaminase-induced a synuclein aggregation (PNAS, February 18, 2003, Vol. 100, no. 4, pp 2047-2052), but are not limited thereto. The present invention is applicable to the treatment of all diseases caused by the overexpression of transglutaminase in nerve tissues.

As for cancers, these are found to significantly increase in the level of expression of transglutaminase upon metastasis or entry into chemo- or radio-resistance. Thus, the suppression of transglutaminase arises as a key in the prophylaxis and treatment of cancers. Concrete examples of the cancers, which can be prevented or treated using the pharmaceutical composition containing chlorogenic acid of the present invention, include large intestine cancer, small intestine cancer, rectal cancer, anal cancer, esophageal cancer, pancreatic cancer, stomach caner, kidney cancer, uterine cancer, breast cancer, lung cancer, lymphoma, thyroid cancer, prostatic carcinoma, leukemia, skin cancer, colon cancer, encephaloma, bladder cancer, ovarian cancer, and gallbladder carcinoma, but are not limited thereto.

The composition comprising chlorogenic acid, a pharmaceutically acceptable salt thereof, or a derivative thereof, and the treatment method of the present invention can be applied to mammals that may suffer from diseases due to the activation of transglutaminase, including cattle, horses, sheep, pigs, goats, camels, antelopes, dogs, and cats, as well as humans.

The pharmaceutical composition comprising chlorogenic acid, a pharmaceutically acceptable salt thereof, or a derivative thereof of the present invention may be used alone or in combination with other pharmaceutical compositions.

The pharmaceutical composition comprising chlorogenic acid, a pharmaceutically acceptable salt thereof, or a derivative thereof may be formulated into various dosage forms. For example, it may be loaded into a capsule containing chlorogenic acid without an excipient or together with a fine solid carrier and/or a liquid carrier. If necessary, the resultant may be molded into preferred formulations. Examples of the suitable carriers include starch, water, brine, ethanol, glycerol, Ringer's solution, and dextrose solutions. Reference may be made to the literature (Remington's Pharmaceutical Science, 19^{th}Ed., 1995, Mack Publishing Company, Easton Pa.) upon formulation of the pharmaceutical composition.

The pharmaceutical composition comprising chlorogenic acid, a pharmaceutically acceptable salt thereof, or a derivative thereof of the present invention may be prepared into any dosage form, either oral or non-oral formulation, containing chlorogenic acid, a pharmaceutically acceptable salt thereof, or a derivative thereof as an active ingredient. Non-oral dosage forms may be injections, coatings, and sprays such as aerosols, with preference for injections or sprays such as aerosols. Also preferable are oral dosage forms.

Examples of the oral dosage forms suitable for the pharmaceutical composition of the present invention include tablets, troches, lozenges, aqueous or emulsive suspensions, powder, granules, emulsions, hard or soft capsules, syrups, and elixirs. For formulation such as tablets and capsules, useful are a binder such as lactose, saccharose, sorbitol, mannitol, starch, amylopectin, cellulose and gelatin, an excipient such as dicalcium phosphate, a disintegrant such as corn starch and sweet potato starch, and a lubricant such as magnesium stearate, calcium stearate, sodium stearylfumarate, and polyethylene glycol wax. For capsules, a liquid carrier such as lipid may be further used in addition to the above-mentioned compounds.

For non-oral administration, the pharmaceutical composition of the present invention may be formulated into injections for subcutaneous, intravenous, or intramuscular routes, suppositories, or sprays inhalable via the respiratory tract, such as aerosols. Injection preparations may be obtained by dissolving or suspending chlorogenic acid, a pharmaceutically acceptable salt thereof, or a derivative thereof, together with a stabilizer or a buffer, in water and packaging the solution or suspension in ampules or vial units. Suppositories are typically made of a suppository base, such as cocoa butter and another glyceride, or a therapeutic laxative. For sprays, such as aerosol, a propellant for spraying a water-dispersed concentrate or wetting powder may be used in combination with an additive.

The pharmaceutical composition of the present invention may be administered via typical routes, such as rectal, local, intravenous, intraperitoneal, intramuscular, intraarterial, transdermal, intranasal, inhalational, intraocular, and subcutaneous routes. Non-oral administration means administration modes including intravenous, intramuscular, intraperitoneal, intrasternal, transdermal and intraarterial routes. For administration via non-oral routes, the pharmaceutical composition comprising chlorogenic acid in a desired purity is preferably mixed with a pharmaceutically acceptable carrier, that is, a carrier being non-toxic at dosage concentrations and amounts, and compatible with other ingredients, and then formulated into a unit dosage form. In particular, it is required to exclude oxidants and other compounds known to be hazardous to the human body.

The chlorogenic acid, a pharmaceutically acceptable salt thereof, or a derivative thereof of the present invention may be administered along with at least one pharmaceutically acceptable excipient as a pharmaceutical composition. It will be obvious to those skilled in the art that when the pharmaceutical composition of the present invention is administered to human patients, the total daily dose should be determined through appropriate medical judgment by a physician. The therapeutically effective amount for patients may vary depending on various factors well known in the medical art, including the kind and degree of the response to be achieved, concrete compositions according to whether other agents are used therewith or not, the patient's condition such as age, body weight, state of health, sex, and diet, the frequency, time and route of administration, the secretion rate of the composition, the time period of therapy, etc. For agents suitable for use in the art, reference may be made to the literature (Remington's Pharmaceutical Science, 19^{th}Ed., 1995, Mack Publishing Company, Easton Pa.). Accordingly, the effective dosage of chlorogenic acid, a pharmaceutically acceptable salt thereof, or a derivative thereof is preferably determined with reference to the above-mentioned considerations.

Hereinafter, a better understanding of the present invention may be obtained through the following examples which are set forth to illustrate, but are not to be construed as the limit of the present invention.

### Mode for the Invention

### Example 1

### In Vitro Assay for Inhibition of Transglutaminase Activity

To measure the inhibitory activity of chlorogenic acid which competes with putrescine, the transglutaminase-catalyzed reaction between [1,4,-¹⁴C] putrescine and succinylated casein were observed.

Succinylated casein was purchased from Calbiochem (Cat. No. 573464), and 1 g of the powder was dissolved in 50 ml of a reaction buffer solution containing 5 mM DTT (0.1 M Tris-acetate (pH 8.0), 10 mM CaCl, 0.15 M NaCl, 1.0 mM EDTA). This solution was stored in a deep freezer for further use. [1,4-¹⁴C] Putrescine dihydrochloride was purchased from GE Healthcare (Cat. No. CFA301), and the stock solution was diluted with distilled water to yield a radiological dose of 5 μCi/ml. Transglutaminase 2 was purchased from Sigma-Aldrich (Cat. No. T5398), and diluted with distilled water to yield a final concentration of 1 unit/ml. Chlorogenic acid (Sigma-Aldrich, Cat No. C3878) was dissolved in DMSO at a concentration of 10 mM to prepare its stock solution, and the stock solution was diluted with DMSO to prepare solutions having various concentrations.

450 μl of succinylated casein solution and 50 μl of [1,4-¹⁴C] putrescine dihydrochloride solution were mixed together to prepare a substrate solution. 96 μl of the reaction buffer, 3 μl of the stock solution of chlorogenic acid, and 1 μl of the stock solution of transglutaminase were mixed together to prepare each sample, followed by incubation at 37° C. for 10 min. 500 μl of the substrate solution and 100 μl of sample solution were mixed well, and the mixture was incubated at 37° C. for 2 hrs, before termination with 4.5 ml of cold (4° C.) 7.5% TCA. The final solution was stored at 4° C. for 1 hr. The TCA-precipitates were filtered through a GF/glass fiber filter, washed with 25 ml of cold 5% TCA, and dried. Radioactivity of crosslinked protein was measured using a β-counter (Beckman Coulter), and compensated by the activity of DMSO-control group as a standard. The activity of transglutaminase was represented by the measured values. The assay was repeated three times under the same conditions, and the results are shown in the following Table 1.

TABLE 1

| | | | | | SD (Standard |
|---|---|---|---|---|---|
| Concentration | Assay 1 | Assay 2 | Assay 3 | Mean | deviation) |
| 0.0 μM | 1.0000 | 1.0000 | 1.0000 | 1.0000 | 0.0000 |
| 0.5 μM | 0.9450 | 0.8658 | 0.9028 | 0.9045 | 0.0396 |
| 1.0 μM | 0.8294 | 0.7454 | 0.5411 | 0.7053 | 0.1483 |
| 2.0 μM | 0.4526 | 0.5111 | 0.3081 | 0.4240 | 0.1045 |
| 3.0 μM | 0.2041 | 0.1523 | 0.1209 | 0.1591 | 0.0420 |
| 5.0 μM | 0.1094 | 0.1009 | 0.0797 | 0.0966 | 0.0153 |
| 10.0 μM | 0.0713 | 0.0876 | 0.0754 | 0.0781 | 0.0085 |
| 50.0 μM | 0.0711 | 0.0808 | 0.0703 | 0.0741 | 0.0058 |

In addition, the mean value was depicted in terms of concentration of chlorogenic acid. IC₅₀ values were calculated by a general nonlinear regression method, determined as 1.4915(±0.1229) μM.

The relative inhibition activities of chlorogenic acid against transglutaminase are depicted in FIG. 1. As shown in FIG. 1, the activity of transglutaminase is inhibited in a chlorogenic acid concentration-dependent manner.

### INDUSTRIAL APPLICABILITY

The present invention provides a transglutaminase inhibitor and a method of inhibiting transglutaminase, both of which are based on using chlorogenic acid, a pharmaceutically acceptable salt thereof, or a derivative thereof as an active ingredient.

Featuring the use of chlorogenic acid, a pharmaceutically acceptable salt thereof, or a derivative thereof, the novel method of inhibiting transglutaminase according to the present invention is safely applied to patients who suffer from the diseases caused by the overexpression of transglutaminase, thereby obtaining an inhibitory effect against transglutaminase without causing side-effects.

1. A transglutaminase inhibitor, comprising a substance selected from the group consisting of chlorogenic acid, a pharmaceutically acceptable salt thereof, and a derivative thereof.

2. The transglutaminase inhibitor according to claim 1, wherein the salt of chlorogenic acid is sodium chlorogenate or potassium chlorogenate.

3. A method of inhibiting an activity of transglutaminase using a substance selected from the group consisting of chlorogenic acid, a pharmaceutically acceptable salt thereof, and a derivative thereof.

4. A pharmaceutical composition for the treatment or prevention of a disease caused by the activation of transglutaminase, comprising a substance selected from the group consisting of chlorogenic acid, a pharmaceutically acceptable salt thereof, and a derivative thereof.

5. The pharmaceutical composition according to claim 4, wherein the salt of chlorogenic acid is sodium chlorogenate or potassium chlorogenate.

6. (canceled)

7. (canceled)

8. A method for the treatment or prevention of a disease caused by the activation of transglutaminase, comprising the step of administering the pharmaceutical composition of claim 4 or claim 5.

9. The method according to claim 8, wherein the disease is cancer or a neurological disease.

10. The method according to claim 8, wherein the disease is a neurological disease selected from Alzheimer's disease, Huntington's disease, or Parkinson's disease.
